(19) United States
(12) Patent Application Publication
(10) Pub. No.: US 2003/0051730 A1
(43) Pub. Date: Mar. 20, 2003

(54) Demand supply oxygen delivery system

(76) Inventor: Ross Thuener, Strongsville, OH (US)

Correspondence Address: PEARNE & GORDON LLP, 526 SUPERIOR AVENUE EAST, SUITE 1200, CLEVELAND, OH, 44114-1484, US

(21) Appl. No.: 09/952,704

(22) Filed: Sep. 14, 2001

(51) Int. Cl. A61M 16/00

(52) U.S. Cl. 128/204.26

(57) ABSTRACT

A demand flow oxygen valve for use with low pressure oxygen sources (about 5 to 75 psi). The inlet of the valve is provided with a diameter index safety system (DISS) oxygen-specific connector. The DISS connector is capable of connecting the inlet of the valve to any oxygen source having a male DISS connector at its outlet. Alternatively, a quick-disconnect connector could be provided to the valve. A single valve may be interchanged among multiple oxygen sources. A method of retrofitting existing oxygen supplies includes steps of providing the demand flow valve having a connector and connecting the valve to the existing oxygen source via the connector. An additional step includes providing a connector adapter if the existing oxygen source does not have a corresponding connector at its outlet.

### BACKGROUND OF THE INVENTION

The present invention relates to an improvement in demand flow oxygen conservers and a method for use thereof.

Conventional oxygen therapy involves delivering oxygen to a patient through a nasal cannula from a source of oxygen at a constant flow. Such systems are wasteful since oxygen is delivered even during exhalation. As a result, oxygen sources, such as compressed oxygen tanks, must be replaced often.

Devices have been developed for selectively delivering oxygen to patients, to conserve the supply of oxygen. In most of these devices, oxygen is delivered in pulses, only during inhalation by the patient.

One such device is disclosed in U.S. Pat. No. 5,881,725 to Hoffman et al. In this device, one tube of a dual element nasal cannula apparatus is attached to the patient to sense a negative pressure, causing a conserver to supply oxygen to the other tube.

Existing devices, however, are built into their respective oxygen sources or are adapted to operate only with a specific source.

### SUMMARY OF THE INVENTION

According to the present invention, an oxygen conservation valve assembly is provided. The valve assembly comprises a demand flow valve capable of delivering oxygen on the basis of inhalation, and a connector provided to an inlet of the demand flow valve, the connector communicating with an inlet of the demand flow valve.

According to another aspect of the present invention, an oxygen delivery system for a patient is provided. The system comprises a first oxygen source having a first outlet connector, a second oxygen source having a second outlet connector, and a demand flow valve capable of delivering oxygen on the basis of inhalation by a patient, the demand flow valve having an inlet connector adapted to alternately connect to the first and second outlet connectors.

### BRIEF DESCRIPTION OF THE DRAWINGS

FIG. 1. is a schematic representation illustrating a demand supply oxygen delivery system according to an embodiment of the present invention.

### DETAILED DESCRIPTION OF THE INVENTION

The present invention comprises a demand flow valve or conserver which is provided separately from and may be used as an oxygen delivery means with a variety of sources of oxygen configured for continuous flow, such as oxygen concentrators and liquid oxygen supplies.

Additionally, the present invention provides the conserver with a releasable connecting means for communicating oxygen between the source and the conserver, so that it may be used with any of a number of oxygen sources. For example, an industry standard diameter index safety system (DISS) connector or fitting. Likewise, a quick-disconnect means such as a quick-disconnect connector or fitting may be supplied to the conserver. The DISS specification is disclosed in Compressed Gas Association pamphlet V-5. Several standard types of quick-disconnect connectors are available which can be reliably connected and disconnected without the use of tools. Other releasable connecting means may also be used.

Further, the present invention involves a method for using the improved conservers to modify or retrofit existing oxygen supplies. According to this method, oxygen supplies already in the possession of a patient, such as oxygen concentrators and liquid oxygen tanks, can be retrofitted to improve their efficiency and/or increase output. These supplies are nearly always provided with a DISS connection which will directly connect to the improved conserver. If the oxygen supply of a patient does not have a DISS connector, an adapter may be provided which will allow the conserver to be use with the oxygen source.

When the conserver is used with a liquid oxygen supply, less oxygen will be used and the supply will not need to be replaced or refilled as often. When the conserver is used with an oxygen concentrator, the concentrator will have time to build up a supply of oxygen between inhalations. Thus, the delivered oxygen concentration will be higher.

Further, a patient may have multiple sources of oxygen. For example, a liquid oxygen supply may be provided which is limited to home use due to its size. A portable oxygen source may be additionally provided for short term use away from the home. Using the conserver of the present invention, only one conserver need be provided. The conserver which is normally connected to the home unit can be easily disconnected. The conserver may then be reconnected to the portable unit and taken with the patient on an outing.

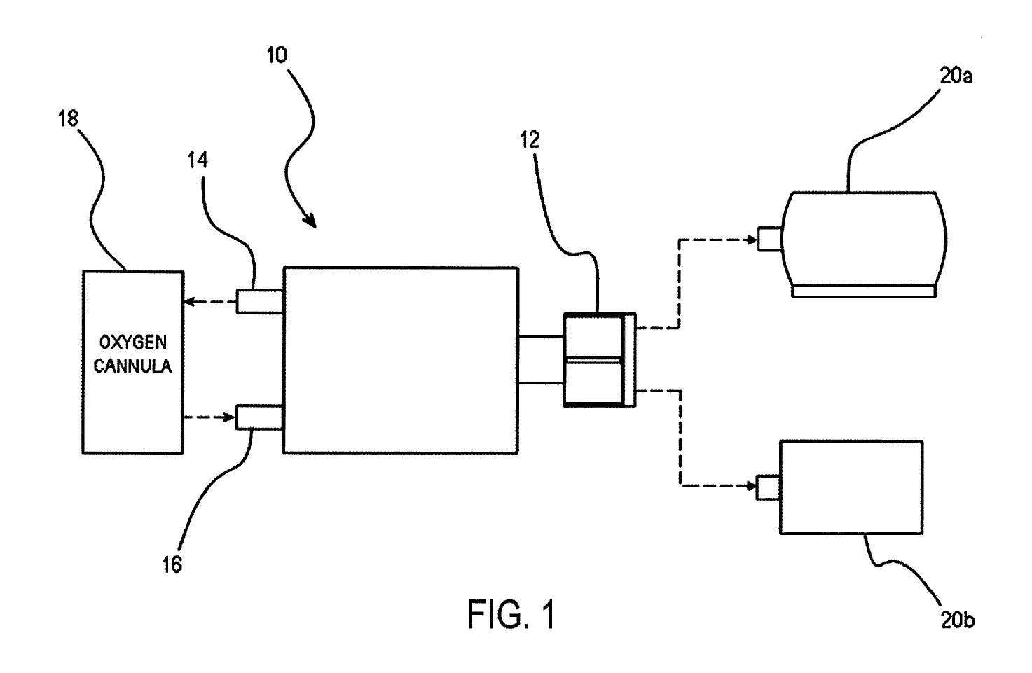

As shown in FIG. 1, an oxygen conserver 10 is shown having a female DISS connector 12 at its input. As shown, the output of the conserver 10 is provided with two barbed fittings or connectors 14, 16 for connecting the tube of a dual element nasal cannula 18. Alternately the conserver 10 could be of the type which operates using only a single tube. Many other devices are known for supplying oxygen on the basis of inhalation which are suitable for use as the oxygen conserver 10 in the present invention.

A low pressure version of the conserver 10, for example, could be designed to receive a supply of oxygen at between 5 and 25 psi at its input. A medium pressure version might be capable of receiving 15 to 75 psi.

If necessary, a regulation means, such as a pressure regulator, is provided within the conserver 10 to provide a constant pressure and/or rate of flow to be output from the conserver 10. The low pressure version, for example, may not require such a regulator.

To connect the conserver 10 to a source of oxygen, such as a low or medium pressure head of a liquid oxygen system 20a or an oxygen concentrator 20b, the source 20a, 20b must first be provided with a male DISS connector. Most stationary oxygen supplies will already be equipped with a DISS connector.

If a male DISS connector is provided on the oxygen supply 20a, 20b, the conserver 10 is directly attached to the male DISS connector of the oxygen supply 20a, 20b using its female DISS connector 12.

In some cases, a different type of connector may be present to the oxygen supply 20a, 20b. In this case, a male DISS connector is then provided by attaching an appropriate adapter to the outlet connector of the oxygen supply 20a, 20b.

Alternatively, a conserver 10′ can be equipped with a quick-disconnect connector 12′. In this way, the conserver 10′ could be exchanged between two or more oxygen supplies more easily.

A quick-disconnect connector 12′ will also allow the conserver 10′ to be used with a central oxygen supply having quick-disconnect outlets located, for example, on the walls of patient rooms in an institutional setting. A single conserver could be used in any room, eliminating the need to have one conserver unit permanently installed in each room of the facility.

Further, the quick-disconnect conserver 10′ can be used with home oxygen supplies, such as those described above, by providing a quick-disconnect adapter similar to the DISS adapter. For example, the conserver 10′ could be easily exchanged between a portable oxygen supply and a stationary supply, each provided with a quick-disconnect adapter.

It should be evident that this disclosure is by way of example and that various changes may be made by adding, modifying or eliminating details without departing from the fair scope of the teaching contained in this disclosure. The invention is therefore not limited to particular details of this disclosure except to the extent that the following claims are necessarily so limited.

What is claimed is:

1. An oxygen conservation valve assembly comprising:
a demand flow valve capable of delivering oxygen on the basis of inhalation; and
a connector provided to an inlet of the demand flow valve, the connector communicating with an inlet of the demand flow valve.

2. The oxygen conservation valve assembly of claim 1 wherein the connector comprises a diameter index safety system connector.

3. The oxygen conservation valve assembly of claim 1 wherein the demand flow valve is adapted to operate with an oxygen source providing oxygen at a pressure of between 5 and 25 pounds per square inch.

4. The oxygen conservation valve assembly of claim 1 wherein the demand flow valve is adapted to operate with an oxygen source providing oxygen at a pressure of between 15 and 75 pounds per square inch.

5. The oxygen conservation valve assembly of claim 1 further comprising a regulator connected between the connector and an outlet of the valve assembly.

6. An oxygen delivery system for a patient, the system comprising:
a first oxygen source having a first outlet connector;
a second oxygen source having a second outlet connector; and
a demand flow valve capable of delivering oxygen on the basis of inhalation, the demand flow valve having an inlet connector adapted to alternately connect to the first and second outlet connectors.

7. The oxygen delivery system of claim 6, wherein each of the connectors are diameter index safety system connectors.

8. The oxygen delivery system of claim 6, wherein each of the connectors are quick-disconnect connectors.

9. The oxygen delivery system of claim 6,
wherein the first oxygen source is selected from an oxygen concentrator and a liquid oxygen supply; and
wherein the second oxygen source is selected from an oxygen concentrator and a liquid oxygen supply.

10. A method of retrofitting a continuous flow oxygen source to provide demand oxygen flow, comprising steps of:
providing a demand flow valve capable of delivering oxygen on the basis of inhalation, the demand flow valve having a connector at an inlet; and
connecting the demand flow valve to an outlet of a continuous flow oxygen source.

11. The method of retrofitting of claim 10, wherein the connector is a diameter safety system fitting.

12. The method of retrofitting of claim 10, wherein the connector is a quick-disconnect connector.

13. The method of retrofitting of claim 10, further comprising the step of adapting the outlet of the continuous flow oxygen source to connect the connector of the inlet.

14. A method of using a demand flow oxygen valve with plural oxygen sources comprising the steps of:
providing a demand flow valve capable of delivering oxygen on the basis of inhalation;
connecting the demand flow valve to a first oxygen source;
disconnecting the demand flow valve from the first oxygen source; and
following the step of disconnecting, connecting the demand flow valve to a second oxygen source.

15. The method of using of claim 14, wherein the first oxygen source is a stationary oxygen source and the second oxygen source is a portable oxygen source.

16. The method of using of claim 14, wherein each of the first and second oxygen sources is selected from one of an oxygen concentrator and a liquid oxygen supply.

17. The method of using of claim 14, further comprising a step of providing an adapter for an inlet of at least one of the first oxygen source and the second oxygen source.

18. The method of using of claim 17, wherein the adapter comprises a diameter index safety system connector.

19. The method of using of claim 17, wherein the adapter comprises a quick-disconnect connector.

20. A kit for adapting a continuous flow oxygen source for demand flow operation, the kit comprising:
a demand flow valve capable of delivering oxygen on the basis of inhalation; and
a connector adapter.

21. The kit of claim 20 wherein the connector adapter comprises a diameter index safety system connector.

22. The kit of claim 20 wherein the connector adapter comprises a quick-disconnect connector.

23. A demand supply means for conserving oxygen delivered to a patient, the valve assembly comprising:
a delivery means for delivering oxygen on the basis of inhalation; and
a releasable connecting means for releasably connection to an inlet of the delivery means.

24. The demand supply means of claim 23 wherein the releasable connecting means comprises a diameter index safety system connector.

25. The demand supply means of claim 23 wherein the releasable connecting means comprises quick-disconnect means for connecting and disconnecting the releasable connecting means without the use of tools.

26. The demand supply means of claim 23 wherein the delivery means operates at an inlet pressure of between 5 and 25 pounds per square inch.

27. The demand supply means of claim 23 wherein the delivery means operates at an inlet pressure of between 15 and 75 pounds per square inch.

28. The demand supply means of claim 23 further comprising a regulation means for regulating at least one of the pressure and flow rate of oxygen, the regulation means being connected between the releasable connection means and an outlet of the demand supply means.

29. A means for delivering oxygen to a patient, the system comprising:
a first oxygen supply means for supplying oxygen, the first oxygen supply means having a first outlet connecting means for providing a connection to a first outlet of the first oxygen supply means;
a second oxygen supply means for supplying oxygen, the second oxygen supply means having a second outlet connecting means for providing a connection to a second outlet of the second oxygen supply means; and
a demand supply means for delivering oxygen on the basis of inhalation, the demand supply means having an inlet connecting means for alternately connecting the demand supply means to the first and second outlet connecting means.

30. The means for delivering oxygen of claim 29, wherein each of the connecting means comprise diameter index safety system connectors.

31. The means for delivering oxygen of claim 29, wherein each of the connecting means comprises quick-disconnect means for connecting and disconnecting the connecting means without the use of tools.

32. The means for delivering oxygen of claim 29,
wherein the first oxygen supply means comprises one of an oxygen concentrator and a liquid oxygen supply; and
wherein second oxygen supply means comprises one of an oxygen concentrator and a liquid oxygen supply.
